(19) United States
(12) Patent Application Publication
(10) Pub. No.: US 2024/0066222 A1
(43) Pub. Date: Feb. 29, 2024

(54) DRUG INFUSION DEVICE WITH EMBEDDED POWER SUPPLY

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/269,963

(22) PCT Filed: Dec. 8, 2021
(86) PCT No.: PCT/CN2021/136514

§ 371 Date: Jun. 28, 2023

(30) Foreign Application Priority Data
Jan. 5, 2021 (WO) PCT/CN2021/070207
Sep. 10, 2021 (WO) PCT/CN2021/117647

(51) Int. Cl. A61M 5/172; A61M 5/142

(52) U.S. Cl. CPC A61M 5/1723; A61M 5/14248; A61M 2202/0486

(57) ABSTRACT

A drug infusion device with embedded power supply includes a drug reservoir, used for accommodating the drug to be infused, provided with a piston and a screw; a driving wheel, connected with the screw, driving the screw to push the piston forward by rotation; a power supply, used to supply power to the infusion device, including a power supply shell, a battery cell, electrolyte and a cover plate; a frame, used to carry the drug reservoir, the driving wheel, the power supply and electrical connection terminals of specific components, the power supply is electrically connected to the electrical connection terminals of specific components to realize power supply to the infusion device; a case, including an upper case and a lower case, for accommodating the drug reservoir, the driving wheel, the power supply and frame.

### CROSS-REFERENCE TO RELATED APPLICATION

This application claims the priority benefit of PCT application no. PCT/CN2021/070207, filed on Jan. 5, 2021 and PCT/CN2021/117647, filed on Sep. 10, 2021. The entirety of the above mentioned patent applications are hereby incorporated by reference herein and made a part of this specification.

### TECHNICAL FIELD

The present invention mainly relates to the field of medical instruments, in particular to a drug infusion device with embedded power supply.

### BACKGROUND

In a healthy person, the pancreas can automatically monitor the amount of glucose in the blood and automatically secrete the required dosage of insulin/glucagon. However, for diabetic patients, the function of their pancreas has been severely compromised, and the pancreas cannot secrete the required dosage of insulin. Therefore, diabetes mellitus is defined as a metabolic disease caused by abnormal pancreatic function, and it is also classified as one of the top three chronic conditions by the WHO. The present medical advancement has not been able to find a cure for diabetes mellitus. Yet, the best the technology could do is control the onset symptoms and complications by stabilizing the blood glucose level for diabetes patients.

Diabetic patients on an insulin pump need to check their blood glucose before infusing insulin into their bodies. At present, most detection methods can continuously detect blood glucose and send the blood glucose data to the remote device in real-time for the user to view. This detection method is called Continuous Glucose Monitoring (CGM), which requires the detection device to be attached to the surface of the patients' skin, and the sensor carried by the device to be inserted into the interstitial fluid for testing. According to the blood glucose (BG) level, the infusion system mimics an artificial pancreas to fill the gaps of the required insulin amount via the closed-loop pathway or the semi-closed-loop pathway.

However, the power supply of the current infusion device is a button battery. Because the limited storage capacity of a single button battery and the large volume ratio of the battery shell, usually, multiple button batteries are required to meet the working requirements of the infusion device. Therefore, a certain space for multiple button batteries must be reserved in the infusion device, leads to the low volume utilization rate of the infusion device, which increases the design difficulty for miniaturization of the infusion device.

Therefore, in the prior art, there is an urgent need for a drug infusion device with a power supply with a smaller volume and a larger capacity.

### BRIEF SUMMARY OF THE INVENTION

The invention discloses a drug infusion device with embedded power supply, the power supply shell and the frame are integrated and/or the cover plate and the lower case are integrated. The shape and size of the power supply are no longer restricted by the shape and size of the button battery shell, and there is no need for a separate shell. The intergrated power supply occupies a small volume, and more active materials can be accommodated to increase the battery capacity, which can further reduce the volume of the infusion device while meeting the long-term working requirements of the infusion device, and improve the user experience.

The invention discloses a drug infusion device with embedded power supply that includes a drug reservoir, used for accommodating the drug to be infused, provided with a piston and a screw; a driving wheel, connected with the screw, driving the screw to push the piston forward by rotation; a power supply, used to supply power to the infusion device, including a power supply shell, a battery cell, electrolyte and a cover plate; a frame, used to carry the drug reservoir, the drive wheel, the power supply and electrical connection terminals of specific components, the power supply is electrically connected to the electrical connection terminals of specific components to realize power supply to the infusion device; a case, including an upper case and a lower case, for accommodating the drug reservoir, the drive wheel, the power supply and frame, the power supply shell is integrated with the frame and/or the cover plate is integrated with the lower case.

According to one aspect of the present invention, an electrolyte isolation layer is arranged on the inside of the power supply shell and the cover plate.

According to one aspect of the present invention, the electrolyte isolation layer is a coated TPE or PET layer.

According to one aspect of the present invention, the thickness of the electrolyte isolation layer is 300 μm-500 μm.

According to one aspect of the present invention, the electrolyte isolation layer is a separated TPE or PET layer.

According to one aspect of the present invention, the battery cell includes a positive electrode sheet, a negative electrode sheet, a separator, a positive electrode tab and a negative electrode tab. The positive electrode tab is fixedly connected to the positive electrode sheet, the negative electrode tab is fixedly connected to the negative electrode sheet, and the electrical connection terminals of specific components includes a plurality of conductors.

According to one aspect of the present invention, the conductors are elastic conductors.

According to one aspect of the present invention, two small holes are provided on the power supply shell, and the positive electrode tab and the negative electrode tab are electrically connected to the plurality of elastic conductor, respectively, through the small holes.

According to one aspect of the present invention, the power supply shell where the small hole located is coated with an insulating sealing material.

According to one aspect of the present invention, when the cover plate is covering on the power supply shell, a part of the positive electrode tab and the negative electrode tab are reserved out of the power supply shell to be electrically connected to the plurality of elastic conductors, respectively.

According to one aspect of the present invention, the connection between the power supply shell and the cover plate is coated with an insulating sealing material.

According to one aspect of the present invention, the positive electrode tab and the negative electrode tab are integrated with the corresponding elastic conductor.

According to one aspect of the present invention, the insulating sealing material is hot melt glue or silica gel.

According to one aspect of the present invention, a protrusion is provided on the elastic conductor.

According to one aspect of the present invention, the infusion device includes an infusion mechanism module and a control mechanism module. The drug reservoir, the drive wheel, the power supply, the frame and the case are arranged on the infusion mechanism module.

According to one aspect of the present invention, the infusion mechanism module and the control mechanism module are designed separately, and the control mechanism module can be reused.

According to one aspect of the present invention, the infusion mechanism module and the control mechanism module are electrically connected by an electrical contact.

According to one aspect of the present invention, the infusion mechanism module and the control mechanism module are disposed of in one housing, discarded together after a single-use.

Compared with the prior art, the technical solution of the present invention has the following advantages:

In the drug infusion device with embedded power supply disclosed by the present invention, the power supply shell and the frame are integrated and/or the cover plate and the lower case are integrated. The shape and size of the power supply are no longer restricted by the shape and size of the button battery shell, and there is no need for a separate shell. The intergrated power supply occupies a small volume, and more active materials can be accommodated to increase the battery capacity, which can further reduce the volume of the infusion device while meeting the long-term working requirements of the infusion device, and improve the user experience.

Furthermore, the electrolyte isolation layer is a TPE or PET layer, which can effectively prevent the power supply shell and cover plate from being corroded by the electrolyte.

Furthermore, the power supply shell coated with hot melt glue, on the one hand, it can prevent the electrolyte from leakaging; On the other hand, it is helpful to the self-thermal runaway management of power supply.

Furthermore, the elastic conductor is provided with protrusions to improve the electrical connection reliability between the elastic conductor and the power supply and the specific connection terminal on the circuit board (or three-dimensional circuit).

Furthermore, the positive electrode tab and the negative electrode tab are integrated with the corresponding elastic conductor, which can avoid the poor electrical connection between the elastic conductor and the positive electrode sheet or the negative electrode sheet from affecting the power supply to specific units.

Furthermore, after a single use, the power supply is discarded with the infusion mechanism module, and the power supply is also replaced when the infusion mechanism module or infusion device is replaced, which can make the power supply always maintain a high-performance working state.

### BRIEF DESCRIPTION OF THE DRAWINGS

FIG. 1a and FIG. 1b are schematic top views of the drug infusion device according to two embodiments of the present invention.

FIG. 2a and FIG. 2b are schematic views of the control mechanism module according to an embodiment of the present invention.

FIG. 3a is a schematic view of the infusion mechanism module according to an embodiment of the present invention.

FIG. 3b is a side view of the assembly of the control mechanism module and the infusion mechanism module according to an embodiment of the present invention.

FIG. 3c is a schematic top view of the lower case of the infusion mechanism module according to an embodiment of the present invention.

FIG. 3d is a schematic top view of the lower case of the infusion mechanism module according to another embodiment of the present invention.

FIG. 4a and FIG. 4b are schematic views of the internal mechanism module of the infusion mechanism module according to an embodiment of the present invention, respectively.

FIG. 4c is a cross-sectional view of the power supply in the Y-Y′ direction according to another embodiment of the present invention.

FIG. 5 is a schematic view of the elastic conductor according to an embodiment of the present invention.

### DETAILED DESCRIPTION

As mentioned above, in the prior art, the button battery shell in infusion device occupies a large volume ratio and the battery capacity is small, which leads to low volume utilization rate of the infusion device, and increases the design difficulty of miniaturization of the infusion device.

In order to solve this problem, the present invention provides a drug infusion device, the power supply shell and the frame are integrated and/or the cover plate and the lower case are integrated.

The shape and size of the power supply are no longer restricted by the shape and size of the button battery shell, and there is no need for a separate shell. The intergrated power supply occupies a small volume, and more active materials can be accommodated to increase the battery capacity and the volume utilization rate of the infusion device, which can further reduce the volume of the infusion device while meeting the long-term working requirements of the infusion device, and improve the user experience.

Various exemplary embodiments of the present invention will now be described in detail regarding the figures. The relative arrangement of the components and the steps, numerical expressions and numerical values outlined in the embodiments are not construed as limiting the scope of the invention.

In addition, it should be understood that, for ease of description, the dimensions of the various components shown in the figures are not necessarily drawn in the actual scale relationship; for example, the thickness, width, length or distance of certain units may be exaggerated relative to other mechanism modules.

The following description of the exemplary embodiments is merely illustrative and does not limit the invention its application or use. The techniques, methods, and devices are known to those of ordinary skill in the art and may not be discussed in detail. However, such techniques, methods, and devices should be considered as part of the specification.

It should be noted that similar reference numerals and letters indicate similar items in the following figures. Therefore, once an item is defined or illustrated in a drawing, it will not be discussed further in the following description of the drawings.

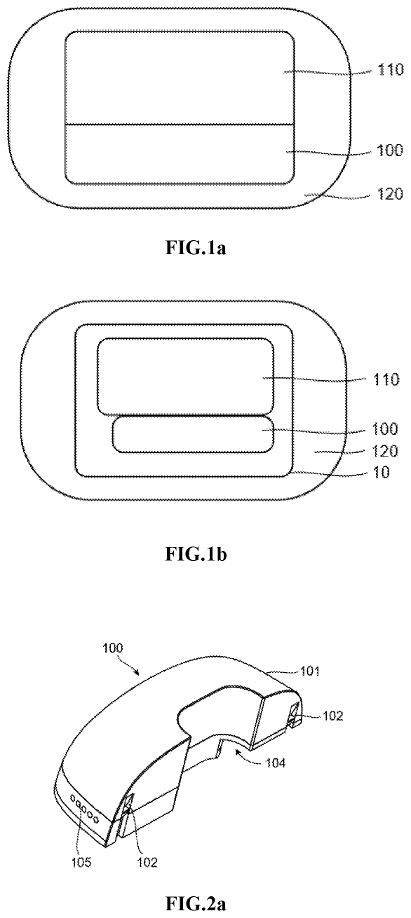

FIG. 1a and FIG. 1b are schematic top views of the drug infusion device according to two embodiments of the present invention.

In the embodiment of the present invention, the drug infusion device with embedded power supply comprises a control mechanism module 100, an infusion mechanism module 110 and an adhesive patch 120, which will be described separately below. In other embodiments of the present invention, the patch-type drug infusion device may include more parts, which are not specifically limited here.

The patch-type drug infusion device refers to a tubing-free infusion device that is entirely pasted on the user's skin surface by the one piece of adhesive patch 120. And the infusion device is provided with an infusion needle unit 121, integrated on the infusion device, instead of a long tube; therefore, the drug can be directly infused from the drug reservoir 131 to the subcutaneous tissue through the infusion needle unit 121.

In the embodiment of the present invention, the infusion mechanism module 110 and the control mechanism module 100 are designed separately and connected by a waterproof plug or directly engaged and electrically connected into a whole. Details regarding how the reliability of the electrical connection has been improved when the infusion mechanism module 110 and the control mechanism module 100 are directly engaged and electrically connected into a whole will be described below. The infusion mechanism module 110 can be reused, and the control mechanism module 100 is discarded after a single use, as shown in FIG. 1a. In another embodiment of the present invention, the infusion mechanism module 110 and the control mechanism module 100 are connected by a wire and disposed of inside the same housing 10. Attached to a certain position of the user's skin by the adhesive patch 120, both units will be discarded together after a single use, as shown in FIG. 1B.

The patch-type drug infusion device of the embodiment of the present invention includes a control mechanism module 100, which receives signals or information from a remote device or a body fluid parameter detection device (such as CGM), and controls the infusion device to infuse drug(s) accordingly.

Inside the housing 101 of the control mechanism module 100 are disposed of program modules, circuit board(s) and related electronic units for receiving signals or issuing control instructions, as well as other mechanical units or components necessary for realizing the infusion function, which is not limited herein. In another embodiment of the present invention, a power supply 133 can also be provided in the control mechanism module. Preferably, in the embodiment of the present invention, the power supply 133 is provided in the infusion mechanism module 110, which will be described below.

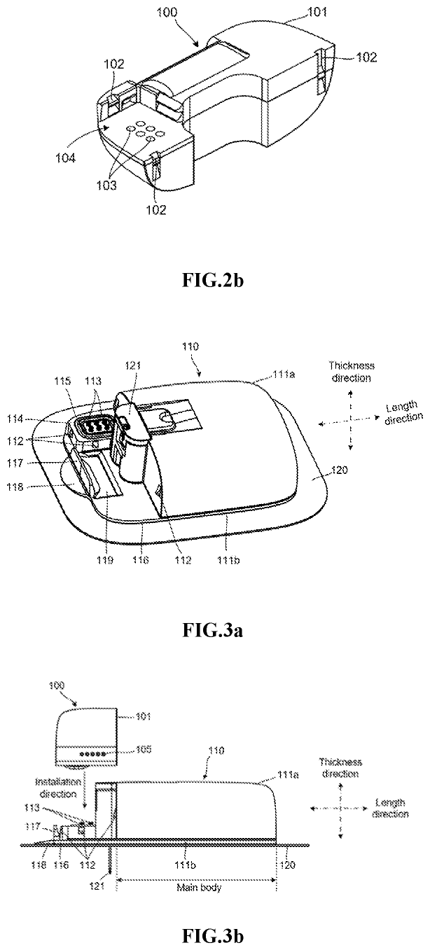

FIG. 2a and FIG. 2b are schematic views of the control mechanism module according to an embodiment of the present invention.

The control mechanism module 100 further includes the first electrical contact 103 exposed on its surface. The first electrical contact 103 is used as a circuit connection terminal to connect the internal circuits provided in the control mechanism module 100 and the infusion mechanism module 110, respectively. The embodiment of the present invention does not specifically limit the positions of the first electrical contact 103.

Compared with the plug connector used as a connection terminal in the prior arts, the contact area of the electrical contact is much smaller, which provides more flexibility to the mechanism module design, and can effectively reduce the volume of the control mechanism module. At the same time, these smaller electrical contact can be directly connected to the internal circuit or electrical components. They could also be directly soldered on the circuit board, which helps to optimize the design of the internal circuit and effectively reduce the complexity of the circuit, thereby saving costs and reducing the volume of the infusion system. Furthermore, the electrical contact is exposed on the surface of the control mechanism module 100 to facilitate electrical connection with connection ends on other mechanism modules. The above technical advantages of the electrical contact is applicable to the first electrical contact 103 on the control mechanism module 100 and the second electrical contact 113 on the infusion mechanism module 110, which will not be described in detail below.

The type of the first electrical contact 103 includes rigid metal pins or elastic conductive members. Preferably, in the embodiment of the present invention, the first electrical contact 103 is a rigid metal pin. One end of the first electrical contact 103 is electrically connected to the connection end provided inside the control mechanism module 100. In contrast, the other end is exposed on the surface of the lower housing 101. The rest part of the first electrical contact 103 is tightly embedded in the housing 101, thus keeping the internal control mechanism module 100 isolated from the outside.

The elastic conductive member includes conductive spring, conductive silica gel, conductive rubber, or conductive leaf spring. One end of the elastic conductive member is used to electrically connect with the internal connection end in the control mechanism module 100, while the other end is used to connect with other connection ends electrically.

As in an embodiment of the present invention, the first electrical contact 103 is a conductive spring. When the electrical contact is in contact with each other, the elasticity of the conductive spring can enhance the reliability of the electrical connection. Similar to the rigid metal pin, one end of the conductive spring is exposed on the surface of the housing 101. In contrast, the remaining part of the conductive spring is tightly embedded in the housing 101 and electrically connected with internal circuits or electrical components. The connection end disposed inside the control mechanism module 100 can be a conductive lead, a specific part of a circuit, or an electrical element.

It should be noted that the “tightly embedded” in the embodiment of the present invention suggests that there is no gap between the electrical contact and the housing 101, keeping the control mechanism module 100 tightly sealed. The following “tightly embedded” has the same meaning as here.

In another embodiment of the present invention, the first electrical contact 103 is a conductive spring, but it is not tightly embedded in the housing 101. Instead, a sealing element is provided in a groove, both of which are disposed around the area where the first electrical contact 103 is located, thus sealing the electrical contact area and the control mechanism module 100.

In the embodiment of the present invention, the control mechanism module 100 is further provided with the first engaging portions 102, which is used to engage the second engaging portion 112 disposed on the infusion mechanism module 110 to assemble the control mechanism module 100 infusion mechanism module 110. Details regarding how the mechanism works to enable the electrical connection between the first electrical contact 103 and the second electrical contact 113 will be described below.

The first engaging portion 102 and the second engaging portion 112 include one or more hooks, blocks, holes, and slots that can be engaged with each other. The positions of the hooks, blocks, holes, and slots can be flexibly adjusted, according to the shape and mechanism module features of the control mechanism module 100 and the infusion mechanism module 110, such as disposed in the interior or on the surface of the corresponding mechanism module, which is not specifically limited herein.

In the embodiment of the present invention, the control mechanism module 100 is further provided with a concave 104 that fits the convex portion 114 disposed at the bottom of the case of the infusion mechanism module 110, which will be described in detail below. Preferably, the first electrical contact 103 is provided in the concave 104, as shown in FIG. 2b.

In the embodiment of the present invention, a buzzer (not shown) is also provided in the control mechanism module 100. When the infusion process starts or ends, the infusion device malfunctions, the drug is exhausted, the control mechanism module 100 issues an error command or receives an error message, etc., the buzzer is used to issue alarm signals, such as sound or vibration, notifying the user to adjust or replace the device in time.

Preferably, in the embodiment of the present invention, the housing 101 of the control mechanism module 100 is provided with a sound-permeable outlet 105 to allow the sound alarm signal from the buzzer to be sent out. In order to achieve a good sealing effect and ensure the normal operation of the buzzer, a waterproof sound-permeable membrane (not shown) is disposed between the sound-permeable outlet 105 and the buzzer. Therefore, the waterproof sound-permeable membrane needs to have a certain porosity to ensure the sound transmission but prevent water molecules penetration.

Compared with the traditional technical solution in which the buzzer is entirely enclosed in the control mechanism module 100, because of the sound-permeable outlet 105, a less loud sound signal emitted from the buzzer would be enough to raise the user's attention, which reduces the energy consumption of the buzzer, thereby optimizing the power consumption configuration of the infusion device and saving production costs.

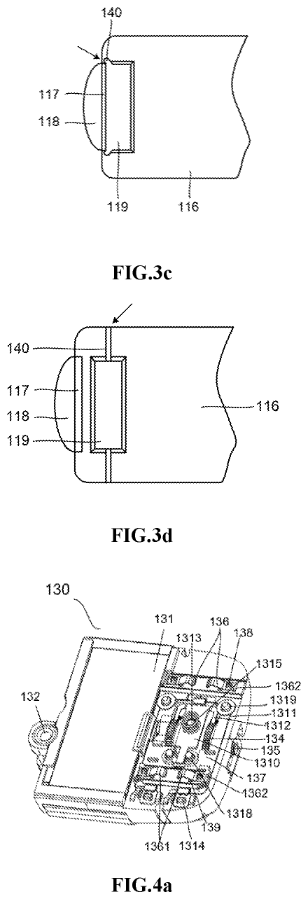

FIG. 3a is a schematic view of the infusion mechanism module 110 according to the embodiment of the present invention. FIG. 3b is a side view of the assembly of the control mechanism module 100 and the infusion mechanism module 110 according to the embodiment of the present invention. FIG. 3c is a schematic top view of the lower case of the infusion mechanism module according to an embodiment of the present invention. FIG. 3d is a schematic top view of the lower case of the infusion mechanism module according to another embodiment of the present invention.

The drug infusion device with embedded power supply further includes an infusion mechanism module 110 with a case. A mechanical module, an electric control module, and other auxiliary modules for completing the drug infusion process are provided inside the case, which will be described in detail below. The case of the infusion mechanism module 110 may include multiple parts. As in the embodiment of the present invention, the case of the infusion system includes an upper case 111a and a lower case 111b.

As mentioned above, in the embodiment of the present invention, the infusion mechanism module 110 is provided with the second engaging portions 112, which is used to engaged and engage the corresponding first engaging portions 102. The positions where the first engaging portions 102 and the second engaging portions 112 are provided correspondingly.

In the embodiment of the present invention, the infusion mechanism module 110 is provided with second electrical contact 113, which are used to press against the corresponding first electrical contact 103 to create an electrical connection between the control mechanism module 100 and the infusion mechanism module 110. The compression between these two corresponding electrical contact disposed on different mechanism modules can improve the reliability of the electrical connection. Similar to the first electrical contact 103, one of the second electrical contact 113 also includes a rigid metal pin and an elastic conductive member. Preferably, in the embodiment of the present invention, the second electrical contact 113 is a conductive spring. Similarly, the conductive spring can improve the electrical connection performance. A groove is also arranged around the area where the second electrical contact 113 is disposed, and a sealing member 115 is arranged in the groove. Similarly, the elasticity of the conductive spring can further improve the electrical connection performance.

Preferably, in the embodiment of the present invention, the two ends of the conductive spring have different diameters. And the diameter of the end exposed to the outside of the infusion mechanism module 110 is shorter than that of the end inside the infusion mechanism module 110. In this way, the conductive spring can be held in the case because of the longer diameter; Thus, when the control mechanism module 100 is not installed on the infusion mechanism module 110, the longer diameter of the inner end can prevent the conductive spring from detaching from the infusion mechanism module 110.

The embodiment of the present invention does not limit the position at where second electrical contact 113 is arranged, as long as it can be electrically connected to the corresponding first electrical contact 103. Preferably, in the embodiment of the present invention, the upper case 111a of the infusion mechanism module 110 includes a convex portion 114 where the second electrical contact 113 is disposed, as shown in FIG. 3a. The shape of the convex portion 114 corresponds to that of the concave 104 disposed on the control mechanism module 100, allowing the two portions to tightly fit each other and press the first electrical contact 103 and the corresponding second electrical contact 113 against each other to realize the electrical connection.

In other embodiments of the present invention, the convex portion 114 may be provided on the lower case 111b. When the infusion mechanism module 110 includes an integral case, the convex portion 114 will be a part of the integral case not specifically limited herein.

The method of assembling the control mechanism module 100 and the infusion mechanism module 110 to each other includes pressing the control mechanism module 100 on the infusion mechanism module 110 along the thickness direction of the infusion mechanism module 110, thereby engaging the first engaging portion 102 and the second engaging portion 112; or pressing the control mechanism module 100 on the infusion mechanism module 110 along the length direction of the infusion mechanism module 110. Alternatively, the control mechanism module 100 can be pressed along with any angle between the thickness direction and the length direction of the infusion mechanism module 110, making the first engaging portion 102 and the second engaging portion 112 engaged with each other. Preferably, in the implementation of the present invention, the method by which the control mechanism module 100 and the infusion mechanism module 110 are assembled is to press the control mechanism module 100 on the infusion mechanism module 110 along with the thickness direction of the infusion mechanism module 110, making the first engaging portion 102 and the second engaging portion 112 engaged with each other, as shown the installation direction in FIG. 3b.

In the embodiment of the present invention, the lower case 111b of the infusion mechanism module 110 further includes an outward extending portion 116. A block 117 is provided outside the outer extending portion 116, as shown in FIG. 3a. As mentioned above, the control mechanism module 100 is pressed to the engaging position along the thickness direction of the infusion mechanism module 110; thus, block 117 can prevent the control mechanism module 100 from detaching along the length direction of the infusion mechanism module 110, ensuring the normal operation of the infusion system. Obviously, in another embodiment of the present invention, if the control mechanism module 100 is pressed to the engaging position along with other directions, the control mechanism module 100 can also be prevented from detaching from the infusion mechanism module 110 by adjusting the position of the block 117.

It should be noted here that “outer” and “outside” are relative to the main body of the infusion mechanism module 110, where they belong to a concept of the relative position, as shown in FIG. 3a or FIG. 3b. The “outside” below have the same meaning as here.

In the embodiment of the present invention, the outer end of the outer extending portion 116 is also provided with a pressing portion 118 for releasing the blocking effect of block 117. While the user is replacing the infusion mechanism module 110, the control mechanism module 100 can be released from block 117 by pressing the pressing portion 118 with a finger. Then, the user can remove the control mechanism module 100 from the infusion mechanism module 110 with two fingers.

Another embodiment of the present invention can also be provided with an unlocking hole 119 disposed of in the inner side of block 117. While the pressing portion 118 is pressed, a finger can enter the unlocking hole 119, thereby pushing the control mechanism module 100 out to separate the control mechanism module 100 from the infusion mechanism module 110. In the embodiment of the present invention, the unlocking hole 119 is square. The square unlocking hole 119 can facilitate the smooth entry of fingers. In other embodiments of the present invention, the unlocking hole 119 may also have other shapes, which is not specifically limited here.

The lower case 111b of the infusion mechanism module 110 is also provided with one or more crease grooves 140. Two crease grooves 140 are provided on both sides of the unlocking hole 119, as shown in FIG. 3c and FIG. 3d. After the crease groove, 140 is provided, the thickness or width of the lower case 111b at the crease groove 140 (as shown by the arrows in FIG. 3c and FIG. 3d) is reduced. When the user presses the pressing portion 118, the lower case 111b is easily broken at the crease groove 140, and the blocking of the control mechanism module 100 by block 117 is more smoothly released.

Preferably, in the embodiment of the present invention, two crease grooves 140 are provided at the both ends of block 117, respectively, as shown in FIG. 3c. In another embodiment of the present invention, the crease groove 140 is provided on two corresponding lateral sides of the unlocking hole 119, as shown in FIG. 3d.

The drug infusion device with embedded power supply further includes a needle unit 121, used for infusing the drug to the subcutaneous tissue.

The adhesive patch 120 is also provided on the bottom of the lower case 111b to attach the infusion device to the user's skin surface.

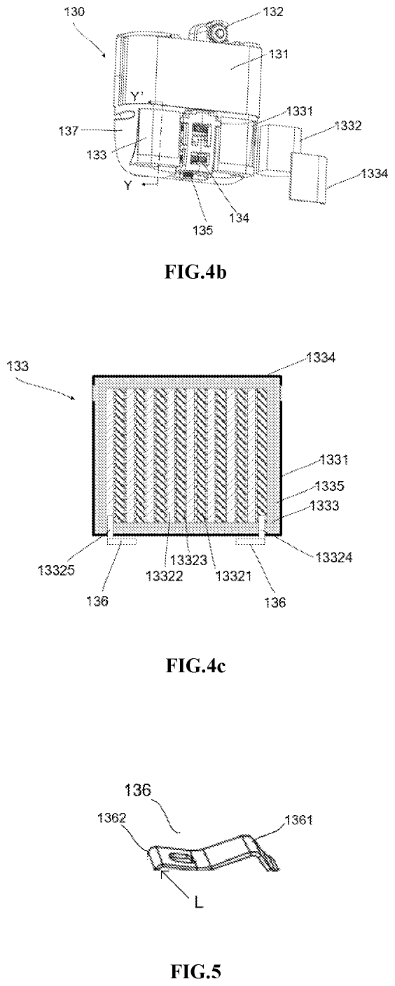

FIG. 4a and FIG. 4b are two schematic views of the internal mechanism module 130 of the infusion mechanism module 110 of the embodiment of the present invention from two perspectives, respectively. FIG. 4c is a cross-sectional view of the power supply in the Y-Y′ direction according to another embodiment of the present invention.

In the embodiment of the present invention, the internal mechanism module 130 includes mechanical units and electronic control units used to realize the infusion function, such as a drug reservoir 131, a drug outlet 132, a power supply 133, a driving wheel 134, a screw 135, a frame 137, a circuit board (not shown), a driving unit 1310, etc. The drug reservoir 131, drug outlet 132, power supply 133, driving wheel 134, screw 135, driving unit 1310 are provided on the frame 137, the frame is also provided with electrical connection terminals of specific components, the power supply 133 is electrically connected to the electrical connection terminals of specific components to realize power supply to the infusion device. The movement of the driving unit 1310 drives the driving wheel 134 to rotate, thus making the screw 135 push the piston (not shown) in the drug reservoir 131 forward, realizing the drug infusion.

In the embodiment of the present invention, the power supply 133 includes a power supply shell 1331, a battery cell 1332, electrolyte 1333 and a cover plate 1334. Put the battery cell 1332 into the power supply shell 1331 and inject the electrolyte 1333 from the opening of the shell 1331, then cover the cover plate 1334, and coat the insulating sealing material at the junction of the cover plate 1334 and the shell 1331. In the embodiment of the present invention, the insulating sealing material is hot melt glue or silica gel. Preferably, the insulating sealing material is hot melt glue, on the one hand, it can prevent the electrolyte from leakaging; On the other hand, it is helpful to the self-thermal runaway management of power supply. In another embodiment of the present invention, the sealing can also be performed in other ways, such as adding a gasket at the cover plate 1334. The specific sealing method is not specifically limited here, as long as the power supply 133 can be sealed to prevent electrolyte from leakaging.

In the embodiment of the present invention, the power supply shell 1331 and the frame 137 of the infusion device are integrated, and the frame 137 is a conventional plastic part, such as PE (polyethylene), PP (polypropylene), PC (polycarbonate)), easy to be corroded by electrolyte, so its inner surface is coated with electrolyte isolating layer 1335, such as spraying PET (polyethylene terephthalate) or TPE (butyl rubber) material, PET and TPE are corrosion-resistant material of electrolyte, which can effectively isolate damage to the power supply shell 1331 and circuit components by the electrolyte. The thickness of the electrolyte isolation layer is 300 μm-500 μm. If the thickness is too thin, the PET film will be infiltrated and softened by the electrolyte. When the amount of electrolyte is small, although the PET film will not dissolve and penetrate, the isolation effect will still exist, but for too long, it may cause the device to deteriorate. While excessive thickness will increase the weight and volume of the power supply shell 1331, which is not conducive to the miniaturization of the infusion device.

In another embodiment of the present invention, the power supply shell 1331 can also be layered, that is, the inner and outer layers are made of different materials, and the outer layer is conventional plastic, such as the aforementioned PE, PP, PC, etc., and the inner layer is TPE (butyl Rubber) or PET (polyethylene terephthalate) layer. TPE is a thermoplastic elastomer material with strong processability and can prevent electrolyte corrosion; PET itself can be used as a container for electrolyte and is resistant to electrolyte corrosion. Both TPE and PET can effectively isolate the electrolyte from damaging the power supply shell and circuit components.

Similarly, the electrolyte isolation layer 1335 is also provided on the inside of the cover plate 1334. Preferably, the electrolyte isolation layer 1335 on the inside of the cover plate 1334 is arranged in the same manner as the power supply housing 1331.

In another embodiment of the present invention, the cover plate 1334 and the upper case 111a of the infusion device (as shown in FIG. 3a) are integrated, and the upper case 111a of the infusion device is a conventional plastic part, such as PE (Polyethylene), PP (polypropylene), PC (polycarbonate), which are easily corroded by electrolyte, so the inner surface is coated with electrolyte isolation layer 1335, such as spraying PET or TPE material, or layered PET or TPE layer.

In the embodiment of the present invention, the power supply shell 1331 and the frame 137, the cover plate 1334 and the upper case 111a may be an integrated at the same time, or may be integrated, respectively. When only one are integrated, for example, when the power supply shell 1331 and the frame 137 are integrated, the cover plate 1334 can be independent of the upper case 111a; when the cover plate 1334 and the upper case 111a are integrated, the power supply shell 1331 can be independent of the upper case 111a. When the power supply shell 1331 and the frame 137, the cover 1334 and the upper case 111a are integrated at the same time, the junction can be coated with insulating sealing material, such as hot melt glue, before the cover plate 1334 is covered on the power supply shell 1331, and after the cover plate 1334 is covered on the power supply shell 1331, the hot melt adhesive can be bonded to the cover plate 1334 and the power supply case 1331 by external heating, such as infrared heating or ultraviolet heating.

In the embodiment of the present invention, the electrolyte 1333 is one of ethylene carbonate, propylene carbonate, diethyl carbonate, dimethyl carbonate, ethyl methyl carbonate, lithium hexafluorophosphate, phosphorus pentafluoride, or hydrofluoric acid.

In the embodiment of the present invention, the material of the separator 13323 is PE (polyethylene) or PP (polypropylene), which may be a single layer of PE or PP, or a three-layer of PE or PP.

In the embodiment of the present invention, the battery cell 1332 is a wound cell or a laminated cell. The specific type of the cell can be selected according to the shape of the power supply shell 1331. When the power supply shell 1331 is cylindrical, the battery cell is a wound cell. When the power supply shell 1331 is square, the battery cell is a square laminated cell. When the power supply shell 1331 is of other special shapes, the corresponding battery cell can also be a special-shaped battery cell. There is no specific limitation, as long as the internal space of the power supply shell 1331 can be fully utilized, the electrode active material is filled to the greatest extent, and increase the battery capacity, so that the capacity of the power supply 133 is increased compared with the button battery, and increase the life time of the infusion device.

The battery cell 1332 includes a positive electrode sheet 13321, a negative electrode sheet 13322, a separator 13323, a positive electrode tab 13324, and a negative electrode tab 13325. One end of the positive electrode tab 13324 is fixedly connected to the positive electrode sheet 13321. Preferably, it is connected by soldering or solder paste, and the other end is electrically connected to an external circuit through a small hole provided in the shell 13321. The specific electrical connection method will be detailed below. The opening and size of the small hole are adapted to the cross-sectional shape and size of the positive electrode tab 13324, and at the same time, an insulating sealing material is coated at the power supply shell where the opening located, to ensure complete sealing and no electrolyte penetration. Preferably, the sealing material is hot melt glue, and the hot melt glue can be helpful to the self-thermal runaway management of the power supply 133 while ensuring complete sealing.

Similarly, one end of the negative electrode tab 13325 is fixedly connected to the negative electrode sheet 13322. Preferably, it is connected by soldering or solder paste, and the other end is electrically connected to an external circuit through a small hole provided on the shell 13321, and at the same time, hot melt glue is coated at the power supply shell where the small hole located.

In another embodiment of the present invention, the positive electrode tab 13324 and the negative electrode tab 13325 may not be electrically connected to the outside through the small hole, but when covering the cover plate 1334, a part of the electrode tab is reserved outside the power supply shell 1331, used for electrical connection with an external circuit, and a sealing material is coated at the junction of the cover plate 1334 and the shell 1331. Preferably, the sealing material is hot melt glue.

In the embodiment of the present invention, the material of the positive electrode tab 13324 is aluminum, and the material of the negative electrode tab 13325 is nickel or copper plated with nickel.

In the embodiment of the present invention, the positive electrode material on the positive electrode sheet 13321 may be manganese dioxide, and the corresponding negative electrode material of 13322 is metal lithium and other lithium-based materials. In other embodiments of the present invention, the positive electrode material may be lithium manganate, lithium cobaltate, lithium iron phosphate and other lithium-containing compounds, the corresponding negative electrode material is graphite.

The infusion mechanism module 110 in the embodiment of the present invention is also provided with a circuit board or multiple three-dimensional circuits coated on the surface of a part of the mechanism module for supplying power to specific units. According to the internal arrangement characteristics of the infusion device, the shape and position of the three-dimensional circuit can be flexibly designed, which can make the full use of the internal space of the infusion mechanism module, making the arrangement more compact. The circuit board is a hard/rigid circuit board or a flexible circuit board. Preferably, in the embodiment of the present invention, the circuit board is flexible. The shape of the flexible circuit board is adjustable, allowing it to be flexibly designed according to the internal space of the infusion mechanism module 110. At the same time, multiple connection ends can be provided on the flexible circuit board to be electrically connected to second electrical contact 113, thereby connecting the circuits of the control mechanism module 100 and the infusion mechanism module 110, allowing the infusion device to perform drug infusion function.

The electrical connection terminals of specific components provided on the frame 137 include electrical conductors, and the electrical conductors include conductive wires, rigid electrical conductors, elastic electrical conductors, and the like. Preferably, the conductor is a plurality of elastic conductors 136, one end of the elastic conductor 136 is electrically connected to the positive electrode tab 13324, preferably, the electrical connection is fixed by soldering, and the other end is connected to the specific connection terminal of the circuit board (or the three-dimensional circuit). One end of the another elastic conductor 136 is electrically connected to the negative tab 13325, and the other end is electrically connected to the specific connection terminal on the circuit board (or the three-dimensional circuit), so as to realize the specific unit power supply.

In another embodiment of the present invention, the positive electrode tab 13324 and the elastic conductor 136 electrically connected thereto are integrated, and the negative electrode tab 13325 and the elastic conductor 136 electrically connected thereto are an integrated. It can be avoided that the poor electrical connection between the elastic conductor 136 and the positive electrode sheet 13324 or the negative electrode sheet 13325, which affects the power supply to the specific unit.

FIG. 5 is a schematic view of the elastic conductor according to an embodiment of the present invention.

In the embodiment of the present invention, at least a protrusion 1361 is provided on the elastic conductor 136, which facilitate the point contact connection or the line contact connection between the elastic conductor 136 and the power supply 133. When the elastic conductor 136 is flat, during use, it is likely to cause a poor connection between the elastic conductor 136 and the power supply 133 and the specific connection end on the circuit board (or three-dimensional circuit), thereby affecting the use effect. In the embodiment of the present invention, protrusion 1361 may be a linear protrusion formed by bending the elastic conductor 136, or it may be several dots or other shapes of protrusions formed by other means on the elastic conductor 136.

In the embodiment of the present invention, an insulating member 1362 is provided on the elastic conductor 136 (as shown at the position L in FIG. 5) to prevent the power unit 1311 from contacting the elastic conductor 136 and causing a short circuit during operation, further stop the infusion mechanism module 130 from working. In the embodiment of the present invention, the insulating member 1362 is formed by printing ink. In other embodiments of the present invention, the insulating member 1362 may also be insulating glue, insulating varnish or insulating material, which is not specifically limited herein.

In the embodiment of the present invention, the frame 137 is further provided with a positioning post 138, the elastic conductor 136 is provided with an opening corresponding to the positioning post 138, and the elastic conductor 136 is sleeved on the positioning post 138 through the opening, so that the elastic conductor 136 is fixed to the frame 137. At the same time, the positioning post 138 is melted by hot melting to further fixed the elastic conductor 136 and to prevent the elastic conductor 136 from shaking due to long-term use or other reasons, which cause the poor electric connection between the elastic conductor 136 and the power supply 133 and the specific connection end on the circuit board (or three-dimensional circuit) and affect the use.

In the embodiment of the present invention, a boss 139 is also provided on frame 137. The boss 139 is located below the elastic conductor 136 to prevent the protrusion 1361 of the elastic conductor 136 from being flattened or deformed during long-term use, which results in a poor electrical connection between the elastic conductor 136 and the specific connection end on the circuit board (or three-dimensional circuit), causing a malfunction. Preferably, the setting position of the boss 139 deviates from the projection position of the protrusion 1361 on the frame 137 and is close to the position post 138. On the one hand, it can prevent the circuit board from being damaged by hard contact or compression between the elastic conductor 136 and the specific connection end on the circuit board (or three-dimensional circuit). On the other hand, it can ensure the elastic electrical contact between the elastic conductor 136 and the specific connection end on the circuit board (or three-dimensional circuit).

Similar to the elastic conductive member above mentioned, the type of the elastic conductor 136 includes a conductive spring, a conductive leaf spring, a conductive rubber, a conductive silica gel, etc., which are not specifically limited herein, as long as they can meet the requirements for electrically connecting the power supply 133 to specific connection ends on the circuit board (or three-dimensional circuit). Preferably, in the embodiment of the present invention, the elastic conductor 136 is the conductive leaf spring.

The elastic conductor 136 can realize the direct electrical connection between the power supply 133 and the specific unit, which reduces the circuit design and the complexity of the internal architecture.

As a summary, the present invention discloses a drug infusion device with embedded power supply, the power supply shell is integrated with the frame and/or the cover plate is integrated with the lower case. The shape and size of the power supply are no longer restricted by the shape and size of the button battery shell, and there is no need for a separate shell. The intergrated power supply occupies a small volume, and more active materials can be accommodated to increase the battery capacity, which can further reduce the volume of the infusion device while meeting the long-term working requirements of the infusion device, and improve the user experience.

While the invention has been described in detail regarding the specific embodiments of the present invention, it should be understood that it will be appreciated by those skilled in the art that the above embodiments may be modified without departing from the scope and spirit of the invention. The appended claims define the scope of the invention.

1. A drug infusion device with embedded power supply, comprising:
a drug reservoir, used for accommodating a drug to be infused, provided with a piston and a screw;
a driving wheel, connected with the screw, driving the screw to push the piston forward by rotation;
a power supply, used to supply power to the drug infusion device, including a power supply shell, a battery cell, electrolyte and a cover plate;
a frame, used to carry the drug reservoir, the drive wheel, the power supply and electrical connection terminals of components, wherein the power supply is electrically connected to the electrical connection terminals of the components to supply power to the drug infusion device; and
a case, including an upper case and a lower case, for accommodating the drug reservoir, the drive wheel, the power supply and the frame, wherein the power supply shell is integrated with the frame and/or the cover plate is integrated with the lower case.

2. The drug infusion device with embedded power supply of claim 1, wherein
an electrolyte isolation layer is arranged on an inside of the power supply shell and the cover plate.

3. The drug infusion device with embedded power supply of claim 2, wherein
the electrolyte isolation layer is a coated TPE or PET layer.

4. The drug infusion device with embedded power supply of claim 3, wherein
a thickness of the electrolyte isolation layer is 300 μm-500 μm.

5. The drug infusion device with embedded power supply of claim 2, wherein
the electrolyte isolation layer is a separated TPE or PET layer.

6. The drug infusion device with embedded power supply of claim 1, wherein
the battery cell includes a positive electrode sheet, a negative electrode sheet, a separator, a positive electrode tab and a negative electrode tab, the positive electrode tab is fixedly connected to the positive electrode sheet, the negative electrode tab is fixedly connected to the negative electrode sheet, and the electrical connection terminal of the components includes a plurality of conductors.

7. The drug infusion device with embedded power supply of claim 6, wherein
the conductors are elastic conductors.

8. The drug infusion device with embedded power supply of claim 7, wherein
two small holes are provided on the power supply shell, and the positive electrode tab and the negative electrode tab are electrically connected to the elastic conductors, respectively, through the two small holes.

9. The drug infusion device with embedded power supply of claim 8, wherein
portions of the power supply shell where the small holes located are coated with an insulating sealing material.

10. The drug infusion device with embedded power supply of claim 7, wherein
when the cover plate covers the power supply shell, a part of the positive electrode tab and a part of the negative electrode tab are reserved out of the power supply shell to be electrically connected to the elastic conductors, respectively.

11. The drug infusion device with embedded power supply of claim 10, wherein
a junction between the power supply shell and the cover plate is coated with an insulating sealing material.

12. The drug infusion device with embedded power supply of claim 8, wherein
the positive electrode tab and the negative electrode tab are integrated with the elastic conductors, respectively.

13. The drug infusion device with embedded power supply of claim 9, wherein
the insulating sealing material is hot melt glue or silica gel.

14. The drug infusion device with embedded power supply of claim 7, wherein
a protrusion is provided on the elastic conductor.

15. The drug infusion device with embedded power supply of claim 1, wherein
the drug infusion device includes an infusion mechanism module and a control mechanism module the drug reservoir, the drive wheel, the power supply, the frame are arranged on the infusion mechanism module.

16. The drug infusion device with embedded power supply of claim 15, wherein
the infusion mechanism module and the control mechanism module are detachable to each other, and the control mechanism module is reusable.

17. The drug infusion device with embedded power supply of claim 16, wherein
the infusion mechanism module and the control mechanism module are electrically connected by an electrical contact.

18. The drug infusion device with embedded power supply of claim 15, wherein
the infusion mechanism module and the control mechanism module are disposed in one housing, discarded together after a single-use.

19. The drug infusion device with embedded power supply of claim 10, wherein
the positive electrode tab and the negative electrode tab are integrated with the elastic conductors, respectively.

20. The drug infusion device with embedded power supply of claim 11, wherein
the insulating sealing material is hot melt glue or silica gel.
